(19) United States
(12) Patent Application Publication
(10) Pub. No.: US 2012/0270949 A1
(43) Pub. Date: Oct. 25, 2012

(54) MELT-GRANULATED CINACALCET

(75) Inventors: Jana Paetz, Bonn (DE); Frank Muskulus, Laupheim (DE)

(73) Assignee: ratiopharm GmbH, Ulm (DE)

(21) Appl. No.: 13/503,048

(22) PCT Filed: Oct. 19, 2010
(86) PCT No.: PCT/EP2010/006390

§ 371 Date: Jul. 3, 2012

(30) Foreign Application Priority Data
Oct. 21, 2009 (EP) 09013308.3

(51) Int. Cl. A61K 31/137; A61K 9/28; A61P 5/18

(52) U.S. Cl. 514/654; 427/2.14

(57) ABSTRACT

The invention relates to an intermediate obtainable by the melt-extrusion of (i) cinacalcet or a pharmaceutically acceptable salt thereof with (ii) a matrix former, and oral dosage forms, especially tablets containing the intermediates of the invention. The invention further relates to a method of preparing the tablets of the invention. Finally, the invention relates to the use of a matrix former and a wicking agent for preparing cinacalcet formulations which can preferably be administered independently of mealtimes.

The invention relates to an intermediate obtainable by jointly melt-processing (i) crystalline cinacalcet or a pharmaceutically acceptable salt thereof, with (ii) a matrix former, and tablets containing the intermediates of the invention. The invention further relates to a method of preparing the tablets of the invention. Finally, the invention relates to the use of a matrix former and a wicking agent for preparing cinacalcet formulations which can preferably be administered independently of mealtimes.

N-[(1R)-1-(1-naphthyl)ethyl]-3-[3-(trifluoromethyl)phenyl]propane-1-amine is known by the INN name “cinacalcet” and has the following structural formula:

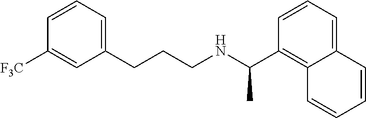

Cinacalcet is a calcimimetic which is used to treat secondary hyperparathyroidism as a consequence of chronic renal failure. In addition, the substance is approved for the treatment of hypercalcaemia in patients with parathyroid carcinoma.

The synthesis and effect of cinacalcet are described in EP 1 203 761 B 1. Patients with a chronic kidney disease often suffer from a parathyroid hyperfunction (secondary hyperparathyroidism) as a consequence of their underlying disease. Failing kidneys excrete less phosphate with the urine and form less active vitamin D3, which is needed in order to maintain a physiological level of calcium ions in the blood. When the level of calcium ions drops, an increased amount of parathyroid hormone is secreted by the parathyroid glands. Overproduction of parathyroid hormone in turn causes calcium ions to be mobilised from the bones and the bones to become more brittle. Cinacalcet binds to the calcium-sensitive receptors on the surface of the parathyroid cells. As a result, the sensitivity of the receptor to extracellular calcium ions is enhanced and a higher calcium level in the blood is simulated than is actually present. As a result of this, the secretion of parathyroid hormone drops, and consequently less calcium is released from the bones.

Cinacalcet is also available in amorphous form by spray-drying, cf. WO 2008/000422 A1. Active agents in amorphous form, however, frequently have disadvantageous properties with regard to their storage stability.

WO 2008/064202 describes compositions containing cinacalcet with delayed release. Dosage forms with delayed release are usually employed for special applications. For a large number of applications, however, dosage forms with immediate release are desirable.

The film-coated tablets currently on the market are tablets with immediate release (=immediate-release tablets) and are described in WO 2005/034928. The tablets contain cinacalcet in micronised form with a content of active agent of about 18%. The film-coated tablets should be taken with or shortly after meals, since the bioavailability is increased by 50 to 80 percent when taken at the same time as food and is only then acceptable.

The micronisation of cinacalcet entails a number of disadvantages, however. First of all, the micronisation results in an active agent with undesirably poor flowability. In addition, the micronised active agent is more difficult to compress, and there is occasionally an uneven distribution of the active agent within the pharmaceutical formulation to be compressed. The considerable enlargement of the surface area during micronisation also causes the sensitivity of the active agent to oxidation to increase.

The objective of the present invention was therefore to overcome the above-mentioned disadvantages. The intention is to provide the active agent in a form which possesses good flowability and makes good compression possible. In addition, it is intended to ensure an even distribution of the active agent. It is intended to avoid micronisation of the active agent.

The intention is also to provide the active agent in a form which possesses good solubility, with good storage stability at the same time. Even after storage for 2 years (or storage for 3 months under stress conditions), correspondingly good solubility ought to be achievable. The intention is to make administration independently of mealtimes possible. The expression “administration independently of mealtimes” is understood to mean that the patient may take the drug with meals, but does not necessarily have to take it at mealtimes. In particular, the aim is to achieve a solubility of greater than 3 mg/ml, especially 10 mg/ml. In addition, it is intended to achieve a storage stability of 12 months at 40° C. and 75% air humidity. The impurities after storage under these conditions are intended to be <2% by weight, especially <1% by weight. Furthermore, it is intended to be possible to provide cinacalcet tablets both with a rapid disintegration time and also with advantageous hardness.

Moreover, it is intended that all the above-mentioned advantageous properties should be achievable with a high proportion of active agent (e.g. with contents of active agent of 20%, 30%, 40% and/or 50%). In particular, it is intended that the above-mentioned properties should also be achievable with a high proportion of active agent and at the same time a high “content uniformity”.

It has been possible to solve the problems of the present invention especially by means of an intermediate which is obtainable by the melt-processing, preferably melt-granulation or melt-extrusion, of cinacalcet and matrix former, and by the use of the intermediate to prepare tablets with immediate release.

The subject matter of the invention is thus an intermediate obtainable by melt-processing
(i) cinacalcet or a pharmaceutically acceptable salt thereof, with
(ii) a matrix former.

As a matter of principle, the term “cinacalcet” (i) in the context of this application comprises both the “free base” described above and also pharmaceutically acceptable salts thereof. These may be one or more salts, which may also be present in a mixture. “Salt” is understood in this context to mean that the amine group of cinacalcet has been protonated, resulting in the formation of a positively charged nitrogen atom, which is associated with a corresponding counter-anion.

The salts used are preferably acid addition salts. Examples of suitable salts are hydrochlorides, carbonates, hydrogen carbonates, acetates, lactates, butyrates, propionates, sulphates, methane sulphonates, citrates, tartrates, nitrates, sulphonates, oxalates and/or succinates.

In the case of cinacalcet, it is particularly preferable that the pharmaceutically acceptable salt should be cinacalcet hydrochloride. It is likewise particularly preferable that the pharmaceutically acceptable salt should be cinacalcet carbonate.

In addition, it is likewise particularly preferable that the pharmaceutically acceptable salt should be cinacalcet methane sulphonate.

The cinacalcet (i) used, preferably the cinacalcet hydrochloride used, will usually be a crystalline material. It has preferably not been micronised. It is preferable for cinacalcet hydrochloride in the polymorphous form I to be used. The polymorphous form I is disclosed, for example, in WO 2007/62147.

The term “non-micronised cinacalcet” refers in the context of this invention to particulate cinacalcet which generally has an average particle diameter (D50) of 20 to 280 μm, preferably 60 to 250 μm, more preferably 65 to 200 μm, even more preferably 70 to 125 and especially 75 to 110 μm.

The expression “average particle diameter” relates in the context of this invention to the D50 value of the volume-average particle diameter determined by means of laser diffractometry. In particular, a Malvern Instruments Mastersizer 2000 was used to determine the particle diameter. All the measuring conditions are selected as described on pages 9 and 10 of WO 2005/034928, i.e. wet measurement, 1,750 rpm, Span® 85 as dispersant, evaluation according to the Fraunhofer method. The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value.

Analogously, the D 10 value of the particle diameter is defined as the particle diameter at which 10% by volume of the particles have a smaller diameter than the diameter which corresponds to the D10 value. Similarly, the D90 value of the particle diameter is defined as the particle diameter at which 90% by volume of the particles have a smaller diameter than the diameter which corresponds to the D90 value.

Furthermore, the non-micronised cinacalcet usually has D 10 values of 1 to 50 μm, more preferably 1 to 30 μm, and especially 2 to 25 μm. In addition, the non-micronised cinacalcet usually has D90 values of 200 to 800 μm, more preferably 250 to 700 μm, and especially 300 to 600 μm.

Crystalline cinacalcet is usually present in the form of needles. Characterisation by means of the volume-average particle diameter may therefore not be specific enough.

It has been found that a more precise characterisation of cinacalcet which can advantageously be used, especially with cinacalcet hydrochloride, can be arrived at by describing the specific surface area.

In a preferred embodiment, (i) crystalline cinacalcet or a pharmaceutically acceptable salt thereof with a specific surface area of 0.01 to 12 m²/g, more preferably 0.1 to 8 m²/g, especially 0.1 to 7 m²/g is used.

The specific surface area is determined in the context of this invention in accordance with the gas adsorption method, especially by means of the BET method.

In a preferred embodiment, the cinacalcet (i) used, especially the cinacalcet hydrochloride, has a water content of 0.01 to 0.20% by weight, more preferably 0.02 to 0.10% by weight. The residual water content is determined according to the Karl Fischer method, using a coulometer at 160° C. A Metrohm 831 KF coulometer with a titration cell without a diaphragm is preferably used. It is usual for a 20 mg sample of cinacalcet to be analysed.

It has been found that a higher water content would have a negative influence on the flowability and hence, in the case of a high content of active agent (drug load), on the uniformity of the content (content uniformity).

The “matrix former” (ii) in the context of this invention is generally a substance which, when heated to above the melting point, especially in a melt-granulation or melt-extrusion process, is deformable and is capable of embedding particulate cinacalcet, i.e. of forming a matrix for particulate cinacalcet. The matrix former thus preferably exhibits thermoplastic behaviour, i.e. it is a thermoplastic matrix former. Furthermore, the matrix former is a substance which is capable of being deposited and accumulating (chemically or physically) on cinacalcet or salts thereof during the extrusion process and of increasing the hydrophilicity of the surface.

The matrix former (ii) may be hydrophilic polymers, especially hydrophilic thermoplastic polymers. This means polymers possessing hydrophilic groups. Examples of suitable hydrophilic groups are hydroxy, amino, carboxy, sulphonate. In addition the hydrophilic polymer which can be used for the preparation of the intermediate preferably has a weight-average molecular weight of 1,000 to 150,000 g/mol, more preferably from 2,000 to 90,000 g/mol, especially 3,000 to 75,000 g/mol. The weight-average molecular weight is preferably determined in the context of this application by means of gel permeation chromatography.

It is preferable that the polymers used as the matrix former should exhibit substantially no emulsifying effect. This means that the matrix former used should preferably not contain any combination of hydrophilic and hydrophobic groups (especially hydrophobic fatty acid groups). In addition, it is preferable for the intermediate of the invention not to contain any polymers that have a weight-average molecular weight of more than 150,000 g/mol. As a rule, polymers of this kind have an undesirable influence on the dissolution characteristics.

When the polymer used as the matrix former is dissolved in water in an amount of 2% by weight, the resulting solution preferably has a viscosity of 0.1 to 8 mPa/s, more preferably 0.5 to 7 mPa/s, especially 1 to 6 mPa/s, measured at 25° C. and determined in accordance with Ph. Eur., 6th edition, chapter 2.2.10.

Furthermore, the hydrophilic polymer used as the matrix former has a glass transition temperature (T_{g}) or melting point of 25° C. to 200° C., more preferably from 40° C. to 170° C. The glass transition temperature is the temperature at which the hydrophilic polymer becomes brittle as it cools down and soft as it heats up. This means that hydrophilic polymers become soft at temperatures above the glass transition temperature (T_{g}) and become plastically deformable without breaking. The glass transition temperature or melting point are determined using a Mettler-Toledo® DSC1, applying a heating rate of 10° C. per minute and a cooling rate of 15° C. per minute. The method of determination is based substantially on Ph. Eur. 6.1, chapter 2.2.34. In order to determine the T_{g}, the polymer is heated twice (i.e. heated, cooled, heated).

In addition, the matrix former (ii) also includes solid, non-polymeric compounds which preferably contain polar side groups.

The intermediate of the invention may, for example, comprise the following hydrophilic polymers as matrix formers: polysaccharides, such as hydroxypropyl methyl cellulose (HPMC), polyvinyl pyrrolidone, polyvinyl alcohol, polymers of acrylic acid and their salts, polyacrylamide, polymethacrylates, vinyl pyrrolidone/vinyl acetate copolymers (such as Kollidon® VA64, BASF), polyalkylene glycols, such as polypropylene glycol or preferably polyethylene glycol, co-block polymers of polyethylene glycol, especially co-block polymers of polyethylene glycol and polypropylene glycol (Pluronic®, BASF), and mixtures of the polymers mentioned.

The matrix former (ii) preferably used is hydroxypropyl methyl cellulose (HPMC), preferably with a weight-average molecular weight of 20,000 to 90,000 g/mol and/or preferably a proportion of methyl groups of 10 to 35%; hydroxypropyl cellulose (HPC), preferably with a weight-average molecular weight of 40,000 to 100,000 g/mol, polyvinyl pyrrolidone, preferably with a weight-average molecular weight of 10,000 to 60,000 g/mol, especially 12,000 to 40,000 g/mol, copolymer of vinyl pyrrolidone and vinyl acetate, especially with a weight-average molecular weight of 40,000 to 75,000 g/mol, polyethylene glycol, especially with a weight-average molecular weight of 2,000 to 50,000 g/mol, polyoxyethylene alkyl ether and/or polyvinyl alcohol, preferably with a weight-average molecular weight of 1,000 to 50,000 g/mol.

Matrix formers (ii) particularly preferably used are co-block polymers of polyethylene glycol and polypropylene glycol, i.e. polyoxyethylene/polyoxypropylene block polymers. These preferably have a weight-average molecular weight of 1,000 to 20,000 g/mol, more preferably 1,500 to 12,500 g/mol, especially 5,000 to 10,000 g/mol. These block polymers are preferably obtainable by condensation of propylene oxide with propylene glycol and subsequent condensation of the polymer formed with ethylene oxide. This means that the ethylene oxide content is preferably present as an “endblock”. The block polymers preferably have a weight ratio of propylene oxide to ethylene oxide of 50:50 to 95:5, more preferably 70:30 to 90:10. The block polymers preferably have a viscosity at 25° C. of 200 to 2,000 mPas, more preferably 500 to 1,500 mPas, especially 800 to 1,200 mPas.

In the context of this invention, it is also possible to use mixtures of the above-mentioned matrix formers. In one possible embodiment, for example, a mixture of polyvinyl pyrrolidone and polyoxyethylene/polyoxypropylene block polymers is used.

In a preferred embodiment, the intermediate of the invention contains cinacalcet or a pharmaceutically acceptable salt thereof, preferably in non-micronised form, and matrix former, wherein the weight ratio of active agent (i) to matrix former (ii) is 5:1 to 1:5, more preferably 3:1 to 1:3, even more preferably 2:1 to 1:2, especially about 1:1.

It is preferable that the type and amount of the matrix former are selected such that at least 50% of the surface area of the resulting intermediate particles is covered with matrix former, more preferably at least 60% of the surface area, particularly preferably at least 80% of the surface area, especially at least 95% of the surface area.

In the context of this invention, it is particularly preferable that cinacalcet (i) and matrix former (ii) are “melt-processed” jointly. It is preferable here that the melt-processing is performed as melt-extrusion or more preferably as melt-granulation. During melt-processing it is also possible for further pharmaceutical excipients, such as disintegrants and wicking agents, to be added, as described below. If disintegrants and wicking agents are contained (more or less intragranularly) in the intermediate of the invention (α), they are referred to in the context of this application as components (iii-int) and (iv-int) respectively. If disintegrants and wicking agents are contained (more or less extragranularly) in the oral dosage form of the invention (β), they are referred to in the context of this application as components (iii-ext) and (iv-ext) respectively.

Hence, the oral dosage form of the invention, preferably in the form of a tablet, preferably with immediate release, may contain:
(α) an intermediate, comprising
   (i) cinacalcet,
   (ii) matrix former,
   (iii-int) disintegrant and/or
   (iv-int) wicking agent; and
(β) pharmaceutical excipients, comprising
   (iii-ex) disintegrant and/or
   (iv-ex) wicking agent.

When reference is made to the total amount of disintegrants and wicking agents (i.e. both extragranular and intragranular), the designations (iii) and (iv) respectively are used.

The melt-processing can be performed, as described below, in conventional melt-processing apparatuses.

The melting conditions when crystalline cinacalcet is used are usually selected such that cinacalcet remains in a crystalline state.

The intermediate of the invention is used in the preparation of an oral dosage form. The oral dosage form is, for example, capsules, powder or granules for filling in sachets or tablets. The preparation of tablets is preferred here. The intermediate of the invention is particularly preferably used for preparing a tablet for immediate release (or simply an “immediate-release tablet”).

The subject matter of the invention is therefore an oral dosage form, especially an immediate-release tablet containing
(α) intermediate of the invention and
(β) pharmaceutical excipients.

These are the excipients (β) with which the person skilled in the art is familiar, especially those which are described in the European Pharmacopoeia.

Examples of excipients (β) used are disintegrants, anti-stick agents, fillers, additives to improve the powder flowability, glidants, wetting agents and/or lubricants.

The ratio of active agent to excipients is preferably selected such that the resulting formulations contain

5 to 60% by weight, more preferably 20 to 45% by weight cinacalcet, and

40 to 95% by weight, more preferably 55 to 80% by weight, pharmaceutically acceptable excipients. As explained above, this is preferably non-micronised, crystalline cinacalcet.

In these ratios specified, the amount of matrix former used to prepare the intermediate of the invention is counted as an excipient. This means that the amount of active agent refers to the amount of cinacalcet contained in the finished formulation.

In a preferred embodiment, the tablet of the invention contains 1 to 40% by weight, 5 to 35% by weight, more preferably 10 to 30% by weight, particularly preferably 15 to 25% by weight disintegrant (iii), based on the total weight of the formulation. “Disintegrants” is the term generally used for substances which accelerate the disintegration of a dosage form, especially a tablet, after it is placed in water. Suitable disintegrants are, for example, organic disintegrants such as carrageenan, celluloses and cellulose derivatives: croscarmellose, starches and starch: derivatives sodium carboxymethyl starch, polysaccharides: soya polysaccharides, alginates and crospovidone. In addition, inorganic disintegrants such as bentonites may be used. Similarly, alkaline disintegrants may be used. The term “alkaline disintegrants” means disintegrants which, when dissolved in water, produce a pH level of more than 7.0. Mixtures of the above-mentioned disintegrants may also be used.

Crospovidone and/or croscarmellose are particularly preferably used as disintegrants, especially in the above-mentioned amounts.

In a preferred embodiment, the tablet of the invention contains 0 to 35% by weight, 1 to 30% by weight, more preferably 5 to 25% by weight, particularly preferably 10 to 20% by weight wicking agent (iv), based on the total weight of the formulation.

Generally speaking, a wicking agent (iv) is an agent with the ability to draw a biological fluid (preferably water) into a solid (preferably into the intermediates (i), preferably by means of physisorption). Physisorption is defined as a form of adsorption in which the fluid molecules can adhere to the surface of the wicking agent, preferably by means of van der Waals binding between the surface of the wicking agent and the adsorbed fluid molecule (preferably water). Normally a wicking agent achieves this with or without swelling. Normally, a non-swelling wicking agent which attracts water will ultimately have a volume consisting substantially of the volume of the wicking agent and the amount of water which it attracts. In general, a swelling wicking agent will have a volume consisting substantially of the volume of the wicking agent, the amount of water which it attracts, and an additional volume, caused by steric and molecular forces.

In the intermediate of the invention or in the tablet of the invention, the wicking agent (iv) preferably causes the formation of channels or pores. This facilitates the penetration of the water molecules into the intermediates, especially by physisorption. The function of the wicking agent therefore consists in transporting water to the surfaces inside the intermediates in order in this way to create channels in or a network on an enlarged surface.

Examples of wicking agents used are: microcrystalline cellulose, silicified microcrystalline cellulose, colloidal silica, kaolin, titanium dioxide, fumed silica, aluminium, niacinamide, m-pyrol, bentonite, magnesium-aluminium silicate, polyester, polyethylene, or mixtures thereof. The wicking agents of the pharmaceutical composition of the present invention preferably contain cellulose and cellulose derivatives, such as silicified microcrystalline cellulose, colloidal silica, and mixtures thereof. The silicified microcrystalline cellulose preferably used is commercially obtainable under the trade name Prosolv® and has a silica content of 1 to 3% by weight, preferably 2% by weight.

The oral dosage form of the invention, especially a tablet, may also contain fillers (v). “Fillers” generally means substances which serve to form the body of the tablet in the case of tablets with small amounts of active agent (e.g. less than 60% by weight). This means that fillers “dilute” the active agents in order to produce an adequate tableting mixture. The normal purpose of fillers, therefore, is to obtain a suitable tablet size.

Examples of preferred fillers are lactose, lactose derivatives, starch, starch derivatives, treated starch, chitin, cellulose and derivatives thereof, calcium phosphate, sucrose, calcium carbonate, magnesium carbonate, magnesium oxide, maltodextrin, calcium sulphate, dextrates, dextrin and/or dextrose, hydrogenated vegetable oil.

Other substances that can be used as fillers are sugar alcohols and/or disaccharides, such as mannitol, sorbitol, xylitol, isomalt, glucose, fructose, maltose and mixtures thereof. The term “sugar alcohols” in this context also includes monosaccharides.

Fillers are usually employed in an amount from 1 to 80% by weight, more preferably 5 to 50% by weight, especially 20 to 40% by weight, based on the total weight of the formulation.

The tablet of the invention may also contain additives to improve the powder flowability. One example of an additive to improve the powder flowability is disperse silica, e.g. known under the trade name Aerosil®. Preferably, silica is used with a specific surface area of 50 to 400 m²/g, determined by gas adsorption in accordance with Ph. Eur., 6th edition, 2.9.26.

Additives to improve the powder flowability are generally used in an amount of 0.1 to 5% by weight, e.g. 1.5 to 4% by weight, based on the total weight of the formulation.

In addition, lubricants can be used. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate (Pruv®) and/or magnesium stearate.

Lubricants are normally used in an amount of 0.1 to 5% by weight, more preferably 1.0 to 3% by weight, based on the total weight of the formulation.

Anti-stick agents can be used in addition. “Anti-stick agents” are usually understood to mean substances which reduce agglomeration in the core bed. Examples are talcum, silica gel, polyethylene glycol (preferably with 2,000 to 10,000 g/mol weight-average molecular weight) and/or glycerol monostearate.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient. Sorbitol, for example—if used as a filler—, is not also used as a matrix former. Similarly, microcrystalline cellulose—if used as a wicking agent—is not also used as a filler, for example (even though microcrystalline cellulose also causes a filling effect).

In a preferred embodiment, the tablet of the invention contains the following components (based on the total weight of the tablet core):

15 to 40% by weight cinacalcet (i)
15 to 35% by weight matrix former (ii)
5 to 40% by weight, preferably 15 to 40% by weight filler (v)
15 to 35% by weight disintegrant (iii),
0 to 30% by weight wicking agent (iv) and
1 to 4% by weight lubricant.

In an alternative preferred embodiment, the tablet of the invention contains the following components (based on the total weight of the tablet core):

more than 40 to 60% by weight cinacalcet (i)
15 to 35% by weight matrix former (ii)
0 to 10% by weight filler (v)
15 to 35% by weight disintegrant (iii),
0 to 20% by weight wicking agent (iv) and
1 to 4% by weight lubricant.

The tablets of the invention preferably do not contain any polymers that lead to a delayed release. It is especially preferable for the tablets of the invention not to contain any polymers that have a molecular weight of more than 150,000 g/mol.

Another subject matter of the invention is a method of preparing the tablets of the invention, comprising the steps of

(a) providing and preferably mixing (i) crystalline cinacalcet or its pharmaceutically acceptable salts with (ii) a matrix former, and optionally further pharmaceutical excipients;
(b) melt-processing, preferably melt-extruding or especially melt-granulating it into an intermediate;
(c) optionally granulating the intermediate;
(d) compressing the resulting intermediates (preferably the granules resulting from step (c)) into tablets, optionally with the addition of further pharmaceutical excipients; and
(e) optionally film-coating the tablets.

In principle, all the explanations given above on preferred embodiments of the intermediate of the invention also apply to the method of the invention.

In a preferred embodiment, in step (a) of the method of the invention (i) cinacalcet, preferably crystalline cinacalcet, or its pharmaceutically acceptable salts are mixed with (ii) a matrix former and optionally further pharmaceutical excipients (β)—as described above.

As mentioned above, the matrix former preferably does not include any polymer with a weight-average molecular weight of more than 150,000 g/mol. The same applies to the pharmaceutical excipients added in step (a) (and/or also in step (d)) of the method of the invention.

The mixing can be performed in conventional mixers. A Turbula® T 10B (Bachofen AG, Switzerland), for example, is suitable. The mixing time is usually 1 minute to 1 hour, preferably 5 minutes to 20 minutes.

In a preferred embodiment, in step (a)

100% of the cinacalcet used,
100% of the matrix former used,
optionally 20 to 70% of the filler used,
optionally 20 to 70% of the wicking agent used, and
optionally 30 to 70% of the disintegrant used, and
optionally 10 to 40% of the lubricant used
are mixed. The remaining optional amounts of filler, disintegrant and lubricant are subsequently added in step (d).

In step (b) of the method of the invention, the mixture from step (a) is melt-processed into the intermediate of the invention, i.e. preferably melt-extruded or melt-granulated. In the course of the melt-processing (b), cinacalcet (i) is processed with the—preferably thermoplastic—matrix former (ii) in such a way that cinacalcet is embedded in the matrix material. It is preferable in this connection that the melting conditions are selected such that the matrix former is melted or partially melted, but the active agent remains in a solid state. Cinacalcet is preferably used in crystalline form (especially cinacalcet hydrochloride in crystalline form I) and the melting conditions are preferably selected such that the active agent is maintained in crystalline form, especially crystalline form I.

The temperature chosen during the melt processing is preferably from 10° C. below to 10° C. above the melting point of the matrix former, preferably with the proviso that the temperature chosen is at least 10° C. below the melting temperature of the cinacalcet used.

The melt-processing can preferably be carried out as melt-granulation or melt-extrusion.

In a preferred embodiment, melt-granulation is performed. In this case, the melting process is preferably performed by means of an intensive mixer with a heatable jacket unit; a Diosna® P1-6, for example, can advantageously be used. In this context, it is usual for the mixture of components (i) and (ii) to be pre-mixed in a dry state without heating the jacket (=step a) and only heated up in a second step (b) by switching on the heatable jacket, preferably with stirring. The heating is preferably continued until an increase in the power consumption is observed. After that, the mixture is granulated and cooled.

In a preferred embodiment, melt-extrusion is performed. This is a continuous method (independent of batches), where the pre-mixing and granulating are not performed sequentially in time, but rather in one production step. A preferred method of preparing the melt extrudate is melt-extrusion by means of a twin-screw extruder (e.g. Leistritz® micro 18). It is an advantage here that setting a temperature gradient, depending on the matrix former chosen, allows the dwell time of the cinacalcet at high temperatures to be reduced considerably. The temperature gradient is usually between 80-190° C. and is preferably selected such that after processing, the cinacalcet is still present in crystalline form.

In the optional step (c) of the method of the invention, the extruded material is granulated. The granulating may take place before, during or after cooling. The granulating preferably already takes place in the course of the melt-processing. As a result, steps (b) and (c) can also be regarded as one processing step.

In a preferred embodiment, the granulation conditions (in step (b) or step (c)) are selected such that the resulting particles (granules) have a weight-average particle size (D50 value) of 75 to 600 μm, more preferably 120 to 500 μm, even more preferably 150 to 400 μm, especially 200 to 350 μm. The weight-average particle size is determined by means of screen analysis (using a Retsch® AS 2000, amplitude 1.5 sec., interval 10 min., amount of sample 15.8 g).

In addition, the granulation conditions are preferably selected such that the resulting granules have a bulk density of 0.3 to 0.85 g/ml, more preferably 0.4 to 0.8 g/ml, especially 0.5 to 0.7 g/ml. The Hausner factor is usually in the range from 1.02 to 1.3, more preferably from 1.03 to 1.25 and especially from 1.04 to 1.15. The “Hausner factor” in this context means the ratio of tapped density to bulk density. The bulk density and tapped density are determined in accordance with USP 24, Test 616 “Bulk Density and Tapped Density”.

In step (d) of the method of the invention, the granules obtained in step (c) are are pressed into tablets, i.e. the step involves compression into tablets. The compression can be performed with tableting machines known in the state of the art, such as eccentric presses or rotary presses. In the case of rotary presses, a compressive force of 2 to 40 kN, preferably 2.5 to 35 kN, is usually applied. As an example, the Fette® 102i press (Fette GmbH, Germany) is used. In the case of eccentric presses, a compressive force of 1 to 20 kN, preferably 2.5 to 10 kN, is usually applied. By way of example, the Korsch® EK0 is used.

Process step (d) is preferably performed in the absence of solvents, especially organic solvents, i.e. as dry compression.

In step (d) of the method of the invention, pharmaceutical excipients (β) may be added to the intermediates from steps (b) or (c). On this subject, reference may be made to the above explanations on suitable excipients.

The subject matter of the invention is not only the method of the invention, but also the tablets produced by this method.

The tablets produced by the method of the invention may be tablets which can be swallowed unchewed (non-film-coated or preferably film-coated). They may likewise be chewable tablets or dispersible tablets. “Dispersible tablet” in this context means a tablet to be used for producing an aqueous suspension for swallowing.

In the case of tablets which are swallowed unchewed, it is preferable that they be coated with a film layer in step (e) of the method of the invention. The above-mentioned ratios of active agent to excipient, however, relate to the uncoated tablet.

For film-coating, macromolecular substances are preferably used, such as modified celluloses, polymethacrylates, polyvinyl pyrrolidone, polyvinyl acetate phthalate, zein and/or shellack.

HPMC is preferably used, especially HPMC with a weight-average molecular weight of 10,000 to 150,000 g/mol and/or an average degree of substitution of —OCH₃ groups of 1.2 to 2.0.

The thickness of the coating is preferably 1 to 100 μm more preferably 2 to 80 μm.

The tableting conditions are preferably selected such that the resulting tablets have a ratio of tablet height to weight of 0.005 to 0.3 mm/mg, particularly preferably 0.05 to 0.2 mm/mg.

In addition, the resulting tablets preferably have a hardness of 70 to 200 N, particularly preferably 100 to 150 N, especially if the tablet weight is more than 200 mg. If the tablet weight is 200 mg or less, the resulting tablets preferably have a hardness of 30 to 100 N, particularly preferably 50 to 70 N. The hardness is determined in accordance with Ph. Eur. 6.0, section 2.9.8.

In addition, the resulting tablets preferably have a friability of less than 3%, particularly preferably less than 1%, especially less than 0.8%. The friability is determined in accordance with Ph. Eur. 6.0, section 2.9.7.

Finally, the tablets of the invention usually have a content uniformity of 95 to 105% of the average content, preferably 98 to 102%, especially 99 to 101% of the average content. The content uniformity is determined in accordance with Ph. Eur. 6.0, section 2.9.6.

The release profile of the tablets of the invention according to the USP method (paddle, 900 ml 0.1 N HCl, pH 1.2, 37° C., 75 rpm) after 10 minutes usually indicates a released content of at least 30%, preferably at least 50%, especially at least 70%. If these parameters are met, the tablets are regarded as tablets with immediate release.

The above details regarding hardness, friability, content uniformity and release profile preferably relate here to the non-film-coated tablet.

As an alternative to compression into tablets, the granules resulting in step (c) of the method of the invention may also be processed—optionally with the addition of further pharmaceutical excipients—into a particulate dosage form, such as by filling into capsules or sachets.

Hence, the subject matter of the invention is an oral dosage form containing cinacalcet, matrix former and disintegrant for the treatment of hyperparathyroidism, wherein the administration is independent of mealtimes. In a preferred embodiment, a disintegrant is used in an amount of 10 to 30% by weight, based on the total weight of the oral dosage form. In a further preferred embodiment, a polyoxyethylene/polyoxypropylene block polymer is used as the matrix former for this purpose, especially as described in more detail above. In a further preferred embodiment, the content of cinacalcet is 20 to 60% by weight, especially 40 to 60% by weight.

The invention will now be illustrated with reference to the following examples.

### EXAMPLES

### Example 1

### Core:

cinacalcet hydrochloride (D50 101 μm): 33.0 mg
polyoxyethylene/polyoxypropylene block polymer (Mw approx. 8,350): 30.0 mg
sorbitol (filler): 47.0 mg
sodium stearyl fumarate: 7.00 mg
crospovidone: 28.0 mg

### Film:

### Opadry® AMB 6.40 mg

The production process comprised the following steps:
cinacalcet HCl and polyoxyethylene/polyoxypropylene block polymer, 20 mg, were granulated while being heated gently to the melting point of the polymer
the resulting intermediate was screened (mesh width 0.6 mm) and mixed for a further 10 min,
the granules were mixed for 10 min together with crospovidone and sorbitol,
sodium stearyl fumarate was added by screening (mesh width 0.3 mm) and mixed for a further 5 minutes,
the mixture obtained was pressed into tablets (9.7×5 r 3.6; 4.5 kN; 50 N), and
the tablets were coated with an Opadry® AMB solution.

The resulting tablets exhibited advantageous solubility properties, which were maintained after storage for three months (at 40° C., 75% air humidity), cf. Example 2.

### Comparative Example 1

For comparison purposes, tablets in accordance with WO 2005/34928 A1 (paragraph [0057]), containing 30 mg micronised cinacalcet HCl, were produced by means of wet granulation. The solubility behaviour was investigated in Example 2.

### Example 2

The in-vitro solubility behaviour of (non-film-coated) tablets in accordance with Example 1 and Comparative example 1 was investigated in accordance with USP (paddle, 900 ml 0.1 N HCl, pH 1.2, 37° C., 75 rpm) before and after storage (40° C., 75% rel. air humidity).

| | | Amount | Amount | Amount | Amount |
|---|---|---|---|---|---|
| | Stirring | without | after | after | after |
| Example | time | storage | 2 weeks | 4 weeks | 12 weeks |
| Example 1 | 15 | 60.2 | 90.4 | 92.2 | 100.8 |
| Comparative | 15 | 83.1 | 86.5 | 84.3 | 83.7 |
| example 1 | | | | | |

The measurement shows that the tablets of the invention exhibit very good solubility behaviour, especially after storage, and it was also possible to avoid micronisation of the active agent.

### Example 3

Tablets in accordance with Example 2 were investigated before and after storage (40° C., 75% rel. air humidity) to determine the contents and measure the impurities by means of the HPLC method.

HPLC parameters:
column: X-Bridge C18 150×4.6 mm, 3.5 μm,
flow rate: 0.9 ml/min.
column temperature: 60° C.,
injection volume: 2 μl,
eluant A: 25 mmol/l KH₂PO₄*H₂O pH 3.00±0.05
eluant B: acetonitrile
pump gradient:

| time [min] | % B |
|---|---|
| 0 | 25 |
| 3 | 25 |
| 22 | 65 |
| 25 | 25 |

wavelength: 225 nm,
sample solvent: water/acetonitrile 50/50
sample concentration: 450 μg/ml

| | Without | After 4 | After 12 |
|---|---|---|---|
| | storage | weeks. | weeks. |
| content | 96.3 | 95.52 | 96.09 |
| total impurities | 0.03 | 0.07 | 0.09 |

The analysis shows that the tablets of the invention exhibit very good storage stability.

1. An intermediate prepared by melt-processing a composition comprising
(i) cinacalcet or a pharmaceutically acceptable salt thereof; and
(ii) a matrix former.

2. The intermediate as claimed in claim 1, wherein
the cinacalcet or pharmaceutically acceptable salt thereof is crystalline.

3. The intermediate as claimed in claim 2, wherein the melt-processing is performed under melting conditions such that the cinacalcet remains in a crystalline state.

4. The intermediate as claimed in claim 1, wherein the matrix former comprises hydrophilic polymers with a weight-average molecular weight of 1,000 g/mol to 150,000 g/mol.

5. The intermediate as claimed in claim 1, wherein the matrix former comprises polyoxyethylene/polyoxypropylene block polymers, preferably with a weight-average molecular weight of 1,500 g/mol to 12,500 g/mol.

6. The intermediate as claimed in claim 1, wherein the weight ratio of component (i) to component (ii) is 1:5 to 5:1.

7. The intermediate as claimed in claim 1, wherein the composition further comprising
(iii-int) a disintegrant; and/or
(iv-int) a wicking agent.

8. An oral dosage form, preferably in the form of a tablet, preferably with immediate release, comprising
(α) an intermediate in accordance with claim 1; and
(β) a pharmaceutical excipient.

9. The oral dosage form as claimed in claim 8, characterised in that component (β) comprises a disintegrant (iii-ex) and/or a wicking agent (iv-ex).

10. The oral dosage form as claimed in claim 9, wherein the total amount of disintegrants (iii-int) and (iii-ex) is 10 to 30% by weight, based on the total weight of the oral dosage form.

11. The oral dosage form as claimed in claim 8, wherein the oral dosage form is in the form of a tablet and comprises a cinacalcet content of 40 to 60% by weight.

12. A method of preparing an oral dosage form in accordance with claim 8 in the form of a tablet, comprising the steps of
(a) providing a composition comprising (i) cinacalcet or a pharmaceutically acceptable salt thereof with (ii) a matrix former, and optionally further pharmaceutical excipients;
(b) melt-processing the composition into an intermediate;
(c) optionally granulating the intermediate;
(d) compressing the intermediate into tablets, optionally with the addition of further pharmaceutical excipients; and
(e) optionally film-coating the tablets.

13. The method as claimed in claim 12, wherein in step (b) melt-processing is performed under melting conditions such that cinacalcet remains in a crystalline state.

14. The method as claimed in claim 12, wherein granules with a weight-average particle size of 120 to 500 μm are produced in step (b) or (c).

15. An oral dosage form comprising cinacalcet, a matrix former, a wicking agent and a disintegrant for use in a treatment of hyperparathyroidism, wherein the administration is independent of mealtimes.
